(19) United States
(12) Patent Application Publication
(10) Pub. No.: US 2026/0354924 A1
(43) Pub. Date: Nov. 12, 2026

(54) SURGICAL MICROSCOPE DRAPE ASSEMBLY

(71) Applicant: Clear Surgical Solutions, LLC, Naperville, IL (US)

(72) Inventor: Michael C. Phillips, Naperville, IL (US)

(21) Appl. No.: 19/653,491

(22) Filed: Apr. 21, 2026

Related U.S. Application Data
(60) Provisional application No. 63/800,762, filed on May 6, 2025.

(51) Int. Cl. A61B 46/10; A61B 90/20; G02B 21/00; G02B 27/00

(52) U.S. Cl. CPC A61B 46/10; A61B 90/20; G02B 21/0012; G02B 27/0006

(57) ABSTRACT

A drape lens for a drape assembly is provided. The drape lens includes a frame operable to be coupled with a microscope. A lens component is coupled with the frame. A rotatable component is rotatably coupled with the frame. The rotatable component is operable to be coupled with a drape.

### CROSS-REFERENCE TO RELATED APPLICATION

This application claims the benefit of U.S. Provisional Patent Application No. 63/800,762, filed in the U.S. Patent and Trademark Office on May. 6, 2025, which is incorporated herein by reference in its entirety for all purposes.

### FIELD

The present disclosure relates generally to a surgical microscope drape assembly with a drape that is rotatable about the microscope.

### BACKGROUND

Microscopes are often used in operating rooms during surgical procedures. During many of these procedures, such as spinal and cranial procedures, the surgeon must manipulate exceptionally small blood vessels, nerve bundles, muscles, nerves and other tissue. These procedures require viewing the surgical site through a surgical microscope so that the fine details in the surgical site can be clearly seen by the surgeon. However, during an operation, the area around the surgical site must remain sterile. A surgical field, as present in a typical hospital's operating room, is an environmentally controlled area where the risk of infection from naturally occurring organisms, such as bacteria, is minimized. The environment's sterility is thus controlled by limiting the introduction of infection-causing bacteria and other contaminants by maintaining strict control over the personnel and equipment present in an operating room.

### BRIEF DESCRIPTION OF THE DRAWINGS

Implementations of the present technology will now be described, by way of example only, with reference to the attached figures.

FIG. 1 illustrates a system with a microscope and a drape assembly.

FIG. 2A illustrates a perspective view of a microscope.

FIG. 2B illustrates a side view of the microscope.

FIG. 3 illustrates a drape assembly.

FIG. 4A illustrates a bottom view of a lens assembly for the drape assembly.

FIG. 4B illustrates a side view of the lens assembly.

FIG. 4C illustrates a top perspective view of the lens assembly.

FIG. 4D illustrates a front view of the lens assembly.

FIG. 4E illustrates a bottom perspective view of the lens assembly.

FIG. 5 illustrates a diagram of the drape assembly coupling with the microscope.

FIG. 6 is a flow chart of a method to utilize a drape assembly.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

As it is difficult or impossible to fully sterilize the surgical microscope, a sterile drape can be used to cover the microscope. The drape can include a flexible tube of sheet-form material that covers all of the components of the surgical microscope, to include the ocular ports of the microscope head and the structure that supports the head. The drape also can include a transparent window therein for attachment to the objective lens of the microscope. The drape can be manufactured and packaged in a sterile condition so that when it is unpackaged in the operating room and placed on a microscope, it creates a sterile field around the microscope and its components.

Various disadvantages have been realized in trying to adapt sterile drapes to surgical microscopes. One disadvantage arises because many hospitals utilize different configurations of microscopes to perform different types of surgical procedures. Microscopes can be configured to include a variety of ocular view ports in a variety of locations, with such configuration depending on the surgical procedure to be performed. The number and location of ocular ports of a given microscope depends on the surgical procedure taking place and the number of surgeons and or assistants present during the procedure. For example, during cranial operations, surgeons typically stand at the head of the patient side-by-side of one another, with a lead surgeon standing next to one or more assisting surgeons. Thus, with the cranial configuration, the microscope may have up to three ocular ports located approximately side-by-side of one another on one side of the microscope.

This ocular port configuration changes for spinal operations, where surgeons typically stand on opposite sides of a given patient, with a lead surgeon and an assisting surgeon standing preferably 180 degrees from one another on opposite sides of a patient, and up to two assisting surgeons standing on either side of the lead surgeon. With the cranial configuration, the microscope may thus have up to three ocular ports located approximately side-by-side of one another on one side of the microscope and a fourth ocular port located on an opposite side of the microscope, preferably 180 degrees from the lead surgeon's ocular port. In some examples, the fourth position may include a camera.

To effectively cover the microscope, it is usually necessary to place the microscope drape with one or more ocular pockets to accommodate the microscope's ocular ports. Conventionally, the drape is locked into place and orientation once the drape lens is coupled to the microscope’s objective lens barrel. Accordingly, the ocular pockets in the conventional drape are not adjustable to accommodate the different configurations of the ocular ports of the microscope.

The disclosed drape assembly includes a drape lens with a rotatable component. With the rotatable component, the drape can be rotated about the drape lens, and the ocular pockets can be moved to correspond with the configuration of the ocular ports of the microscope. The drape is then not unnecessarily stretched and/or twisted, which may cause damage to the drape and/or provide inadequate coverage of the microscope to maintain the sterile environment.

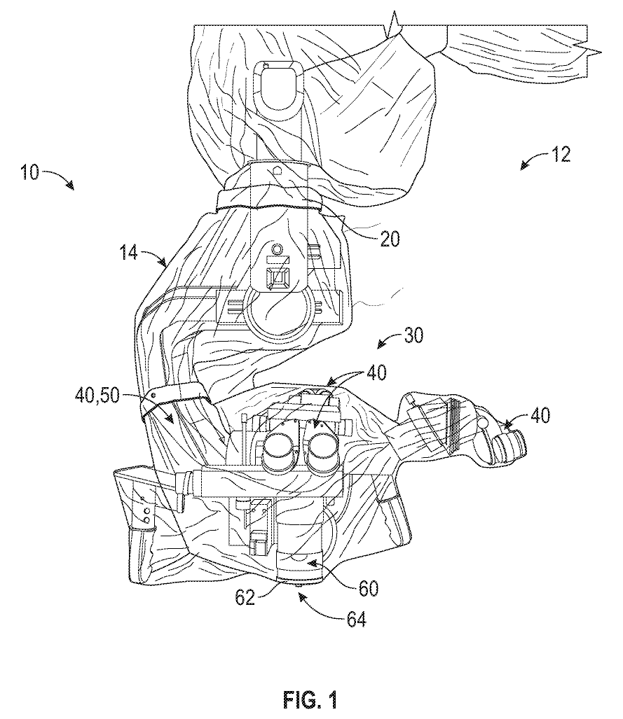

Referring now to FIG. 1, a system 10 is provided. The system 10 can include a microscope 12 with a drape assembly 14 coupled with the microscope 12 to provide a sterile barrier between the microscope 12 and a patient.

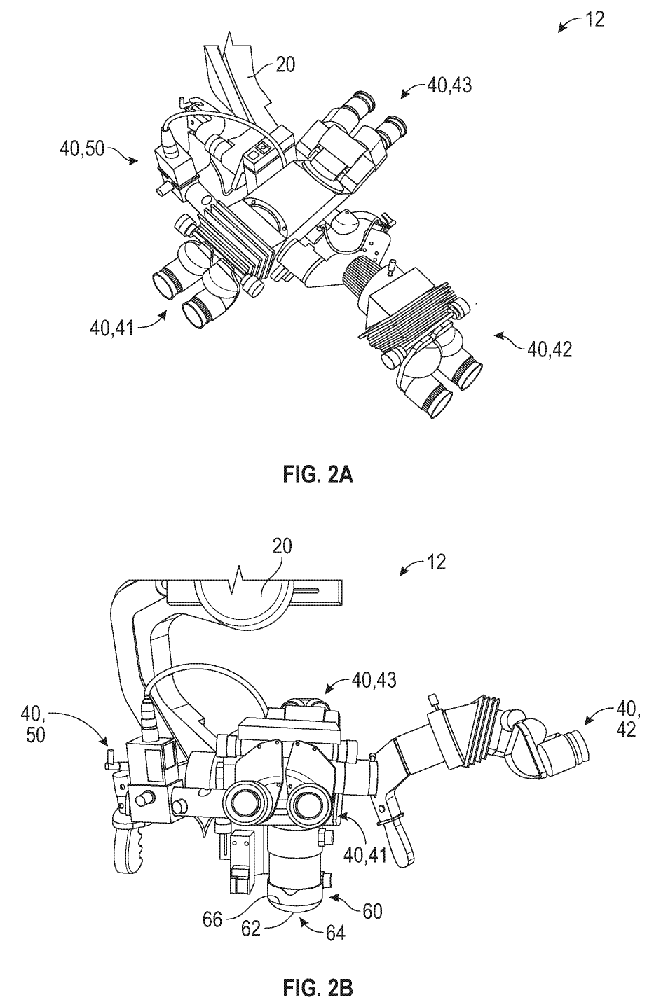

Referring to FIGS. 1, 2A, and 2B, an example microscope 12 is illustrated. In at least one example, the microscope 12 can include a surgical microscope operable to be utilized in an operating room. In some examples, the microscope 12 can be operable to be utilized in a sterile environment. Accordingly, the drape assembly 14 is required to maintain the sterile field.

The microscope 12 can be adjustably mounted relative to a floor, wall, and/or or ceiling mounted structure (not shown) with an articulated support arm 20 supporting a microscope head 30. The microscope head 30, mounted to the free end of the support arm 20, includes at least one ocular port 40, an objective lens barrel 60, and an illumination source (not illustrated) for illuminating the operative field viewed through the microscope 12.

In at least one example, the objective lens barrel 60 can be operable to hold and/or align the objective lens(es), which can be responsible for magnifying and/or focusing the image at different levels. In at least one example, the microscope 12 can include a cavity 64 that can be defined by an inner peripheral wall 62 of the barrel 60.

The ocular ports 40 are operable to allow the operator(s) to view the magnified image or area. The ocular ports 40 can be in communication with the objective lens and/or the objective lens barrel 60. In at least one example, for example as illustrated in FIGS. 1-2B, the microscope 12 can include four ocular ports 40. In other examples, the number of ocular ports 40 can vary. As shown in FIGS. 1-2B, the microscope 12 can include a first ocular port 41, a second ocular port 42, and a third ocular port 43. In some examples, the ocular ports 40 can be arranged such that the first ocular port 41 and the third ocular port 43 extend opposite one another while the second ocular port 42 and the fourth ocular port 50 extends opposite one another while being substantially perpendicular to the first ocular port 41 and the third ocular port 43.

In at least one example, at least one of the ocular ports 40 can allow for the operators to view the magnified image via eyepieces or ocular lenses. For example, as illustrated in FIGS. 1-2B, at least one of the ocular ports 40 (e.g., ocular ports 41, 42, 43) can include eyepieces.

In at least one example, the microscope 12 can also enable a visual tool 50, such as stereoscopic viewing and/or camera attachment. For example, as shown in FIGS. 1-2B, at least one of the ocular ports 40 can include a visual tool 50 instead of and/or in addition to the eyepiece.

During operations, surgeons can stand on opposite sides of a given patient, with a lead surgeon standing on one side of the patient and an assisting surgeon standing on the opposite side of the patient, preferably 180 degrees from or facing the lead surgeon. With this configuration, as illustrated in the microscope of FIGS. 1-2B, the microscope head 30 can have a centrally located first ocular port 41 used by the lead surgeon, with a second ocular port 43 used by the assisting surgeon centrally located, in some examples, 180 degrees from the first ocular port 41. Although the ocular ports 40 of the lead and assistant surgeon are located 180 degrees from one another in at least one example of the microscope 12, the location of the ocular ports 40 from one another may be of any angle that enables the surgeons to stand on opposite sides of the patient.

In some examples, an additional assistant or observer may stand on at least one side of the lead surgeon or assisting surgeon, thus necessitating the use of one or more additional ocular ports 40 (e.g., ocular port 42), with the one or more additional ocular ports 40 being respectively located on each side of the lead or assistant surgeon's respective first or second ocular port 41, 43. With this configuration, three ocular ports 40 can thus be located on one side of the microscope head 30 (one on each side of the centrally located first or second ocular ports 41, 43), with the first and second ocular ports 41, 43 being located, in some examples, about 180 degrees from one another. In some examples, as illustrated herein, a fourth ocular port 40 can be utilized with a visual tool 50 that is about 180 degrees from the third ocular port 42. The angles between ocular ports 40 can vary without deviating from the scope of the disclosure. In some examples, the positioning ocular ports 40 can be moved and/or adjusted so that the angles between ocular ports 40 can vary even during use. Thus, during spinal operations, up to four ocular ports 40 may be used during a given procedure, with three ocular ports 41, 42, 43 being located on one side of the microscope head 30 and patient, and an ocular port 40 with a visual tool 50 and/or with binoculars can be included as a fourth ocular port 40.

In some examples, the arrangement of the ocular ports 40 can be reconfigured so that the placement of the ocular ports 40 can be in differing positions depending on the desired arrangement of the operators and/or visual tool 50. This can be adjusted for any and/or each operation, so even with the same microscope 12 in the same operating room, the arrangements of the ocular ports 40 can be switched about. As such, the orientation and/or arrangement of the drape assembly 14 (e.g., ocular pockets 350) in relation to the microscope 12 (e.g., the barrel 60) may need to be adjusted.

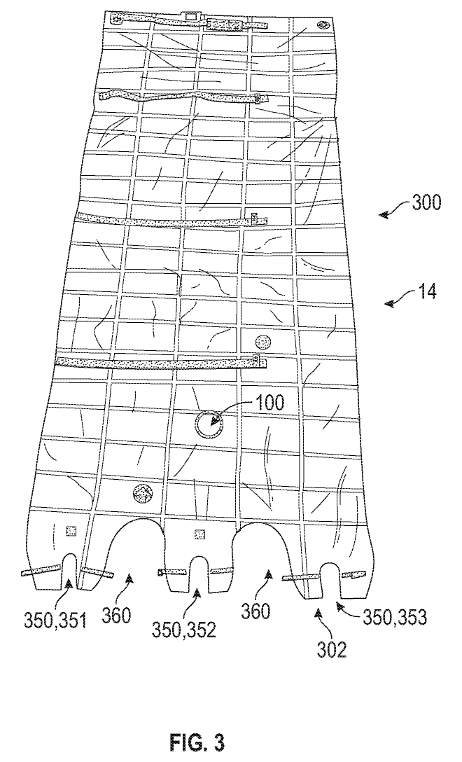

FIG. 3 illustrates a drape assembly 14 that is operable to couple with the microscope 12 to ensure that a sterile environment is maintained during the operation. The drape assembly 14 can include a drape 300 that includes a substantially elongated hollow enclosure made of a sheet-form material. In at least one example, the drape can include a transparent, flexible material, for example made of polyethylene, polypropylene, and/or any other suitable similar polymer or copolymer film of a predetermined thickness. In at least one example, the drape assembly 14 can be provided to the operating room as a sterile object.

A drape lens 100 can be coupled with the drape 300 and positioned within body of the drape 300. The drape lens 100 can be operable to be coupled with the microscope 12 (e.g., the barrel 60) and provide sterile coverage of the barrel 60 while allowing visual access for the microscope 12 through the drape lens 100. As discussed in more detail below, the drape 300 can be rotatably coupled with the drape lens 100 such that the drape 300 is rotatable about the drape lens 100.

In at least one example, the drape 300 can have a first end 302 that can define at least one ocular pocket 350. As illustrated in FIG. 3, the drape assembly 14 can include three ocular pockets 350 to correspond with each of the ocular ports 41, 42, 43 that have eyepieces. In other examples, the drape assembly 14 can include one, two, or more than three ocular pockets 350 so that each of the ocular pockets 350 are operable to correspond with a corresponding ocular port 40 of the microscope 12.

The at least one ocular pocket 350 enable the drape assembly 14 to be used on a variety of microscope heads 30 having a variety of ocular ports 40 placed in a variety of locations. The number and location of ocular ports 40 of a given microscope 12 depends on the surgical procedure taking place and the number of surgeons and/or assistants present during the procedure. For example, during operations such as cranial operations, surgeons can stand at the head of the patient side-by-side of one another, with a lead surgeon standing next to an assisting surgeon. With this configuration, as illustrated in FIGS. 1-2B, the microscope head 30 can have a first ocular port 41, used by the lead surgeon and centrally located on the microscope head 30, and a second ocular port 42, used by the assisting surgeon and located to the right or left of the first ocular port 41. It is noted that a third ocular port 43 may also be utilized during a given procedure, with the third ocular port 43 being located next to the first ocular port 41 on a side opposite the first ocular port 41. Thus, during the operations, up to three ocular ports 40 may be used during a given procedure, with the three ocular ports 40 being located approximately side-by-side of one another on one side of the microscope's head 30. In some examples, as illustrated herein, a fourth ocular port 50 can be included with a visual tool 50, such as a camera. The fourth ocular port 50 can be provided opposite the second ocular port 42.

In at least one example, the drape 300 can be substantially tubular with an opening end opposite the first end 302 to enable the drape assembly 14 to be pulled over the drape of the microscope 12. The opening end can be opened and can be pulled over and past the head 30 of the microscope 12 (for example as shown in FIG. 1) to a location on the microscope 12 away from the head 30 and operative field (for example, where the support arm 20 meets the floor, wall, or ceiling of the operating room). The at least one ocular pocket 350, defined in the first end 302 of the drape 300, can each receive and cover a corresponding ocular port 40 of the microscope 12 when the drape assembly 14 is pulled over the microscope 12 to provide a sterile barrier between the microscope 12 and the patient.

In some examples, as illustrated in FIG. 3, the drape 300 can include recesses 360 between each ocular pocket 350 to accommodate the angle between each ocular port 40 of the microscope 12. Accordingly, the drape 300 is not excessively stretched and/or folded to maintain the sterile environment.

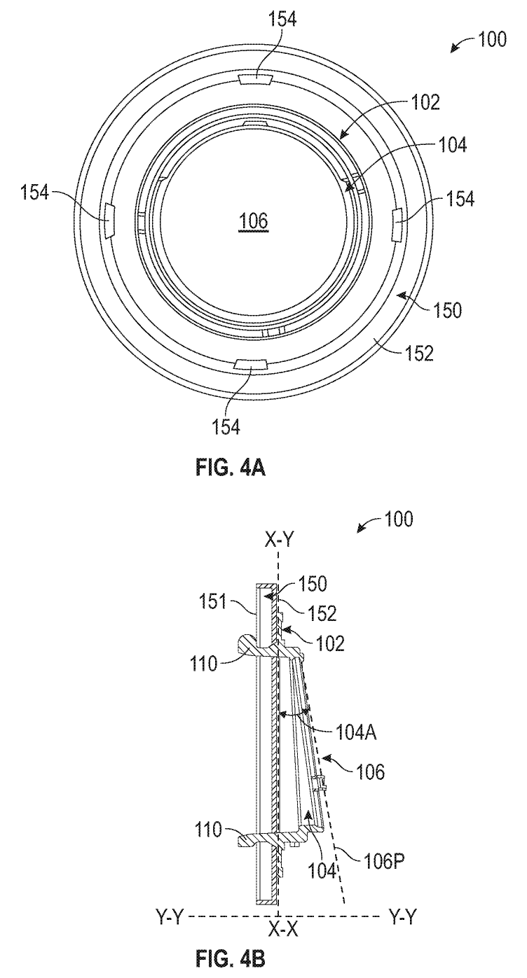

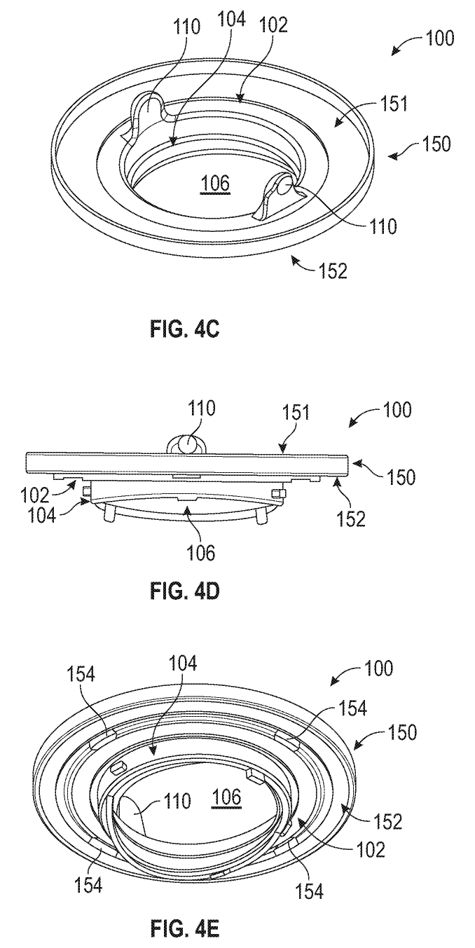

FIGS. 4A-4E illustrate a drape lens 100. The drape lens 100 includes a frame 102 operable to be coupled with the microscope 12.

In at least one example, the frame 102 can be engageable with an inner peripheral wall 62 of the objective lens barrel 60, for mounting the frame 102 on the barrel 60. A lens component 104 can be coupled with the frame 102. The lens component 104 can include a viewing portal 106 operable to permit the objective lens of the microscope 12 to view the patient and/or object through the viewing portal 106. In at least one example, the lens component 104 can be adapted to be located within a cavity 64, defined by the inner peripheral wall 62 of the objective lens barrel 60, when the frame 102 engages the inner peripheral wall 62.

In some examples, the viewing portal 106 of the lens component 104 can be operable to intersect at least one viewing axis (e.g., a pair of viewing axes) defined by the ocular ports 40 of the microscope 12 and an illumination axis defined by an illumination source (not shown) within the microscope 12 when the frame 102 is mounted on the barrel 60. The viewing portal 106 can be acutely inclined at an angle 104A to minimize glare and/or reflection from the illumination source, with the viewing portal 106 having a rotational orientation with the lens barrel to enable an optimum viewability through the viewing portal 106. For example, the viewing portal 106 can extend along a plane 106P that is disposed at the angle 104A relative to a longitudinal plane X-X of the frame 102. In some examples, the angle 104A can be between about 10 degrees and about 2 degrees. In some examples, the angle 104A can be between about 9 degrees and about 5 degrees. In some examples, the angle 104A can be between about 9 degrees and about 7 degrees. In some examples, the angle 104A can be about 8 degrees.

In at least one example, the frame 102 can include at least one holding component 110 that extends therefrom. The holding component(s) 110 can be operable to permit an operator to grasp onto the drape lens 100 while maintaining a sterile environment. For example, as illustrated herein, the frame 102 can include two holding components 110 that extend from the frame 102, away from the viewing portal 106 and the lens component 104. The holding components 110 can include tabs to allow for finger hold of the drape lens 100.

In at least one example, when coupling the frame 102 with the microscope 12, the frame 102 can be received in the cavity 64 and then twisted to be locked in place. In at least one example, the microscope 12 can include a mark 66 that can indicate proper alignment and coupling of the drape lens 100 in relation to the microscope 12 (e.g., the barrel 60). In some examples, the drape lens 100 may include a corresponding mark that can align with the mark 66 when the drape lens 100 is properly aligned and coupled with the microscope 12. In some examples, the alignment of the drape lens 100 is critical to ensure that the viewing portal 106 is aligned with the objective lens of the microscope 12 and ensure optimum viewability through the viewing portal 106. Once the frame 102 is properly aligned and coupled with the microscope 12, the lens component 104 and the drape lens 100 is fixed in position.

As illustrated herein, the drape lens 100 can include a rotatable component 150 that can be rotatably coupled with the frame 102. The rotatable component 150 can be operable to be coupled with the drape 300 such that the drape 300 is rotatable about the drape lens 100 while the lens component 104 and the viewing portal 106 remain stationary.

In at least one example, the rotatable component 150 can include a rotatable washer coupled with the frame 102. In some examples, the rotatable washer 150 can be coupled with the frame 102 via at least one tab 154. In some examples, the tabs 154 can be disposed about a perimeter or a circumference of the frame 102 to provide even distribution of forces onto the rotatable component 150 while allowing for rotation. Accordingly, the rotatable washer 150 can be received in the at least one tab 154 so that the rotatable washer 150 can rotate in relation to and/or about the frame 102.

In at least one example, the rotatable component 150 can include a first side 151 that faces away from the frame 102 and the lens component 104. For example, the first side 151 can face in the same direction that the holding components 110 are extending such that the holding components 110 extend from the frame 102 past the rotatable component 150. The rotatable component 150 can include a second side 152 opposite the first side 151 such that the second side 152 is proximate the lens components 104 and the viewing portal 106.

In at least one example, the drape 300 can be operable to be coupled with at least a portion of the second side 152 of the rotatable component 150 (e.g., opposite the microscope 12). For example, the drape 300 can be heat sealed to the rotatable component 150. The drape 300 can then be operable to be rotatable about the drape lens 100 so that each of the at least one ocular pocket 350 is aligned with the corresponding ocular port 40 of the microscope 12 while the lens component 104 remains stationary.

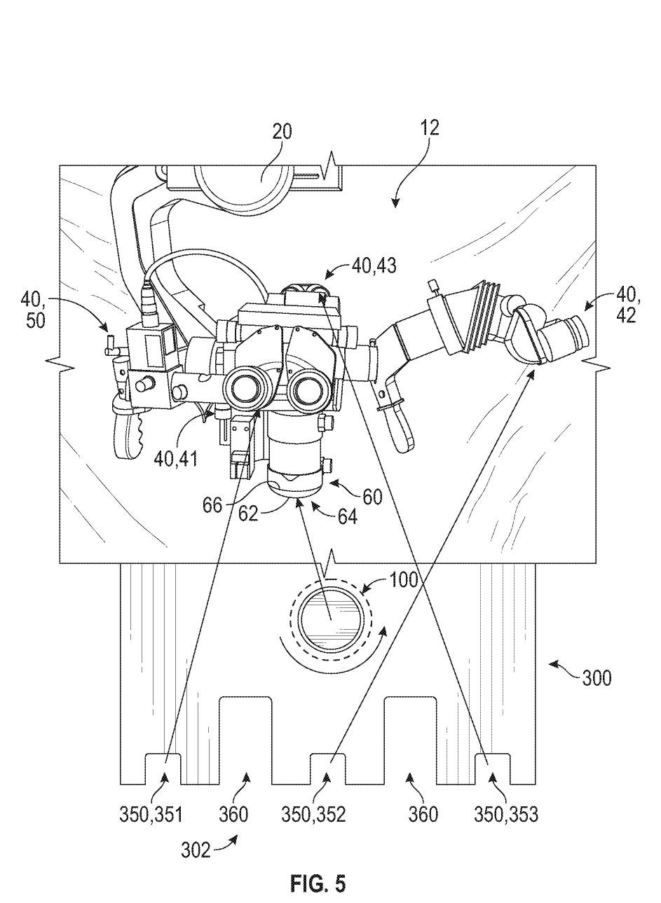

In the exemplary case as illustrated in FIG. 5 where the microscope has three ocular ports 41, 42, 43 with eyepieces, the drape assembly 14 can include three ocular pockets 351, 352, 353 to correspond with the four potential positions of the three ocular ports 41, 42, 43. Even though there is a fourth ocular port 50, the fourth ocular port 50 includes the visual tool 50, and there is not a need for the ocular pocket 350. As the drape 300 is rotatable about the drape lens 100, the configuration and arrangement of the ocular ports 40 can vary, and the drape assembly 14 can simply be adjusted by rotating the drape 300 to correspond with the ocular ports 40. Accordingly, there is not a need for a fourth ocular pocket cut out in the middle of the drape 300 to accommodate the potential of a fourth position of an ocular port,

The ability to rotate the drape 300 as needed to align the ocular pockets 350 to correspond with the ocular ports 40 with eyepieces while keeping the lens component 104 of the drape lens 100 stationary provides the advantage of allowing a single drape assembly 14 to accommodate various surgical procedures, with each procedure having a different ocular port 40 configuration. Additionally, the drape assembly 14 does not have to be stretched and/or twisted to accommodate the configuration of the ocular ports 40. Such a stretching and/or twisting of the drape 300 can compromise the sterile field established by the drape assembly 14 around the microscope 12, and the ability to rotate the drape 300 about the drape lens 100 can allow the drape lens 100 to maintain the quality of the image received by the objective lens of the microscope 12.

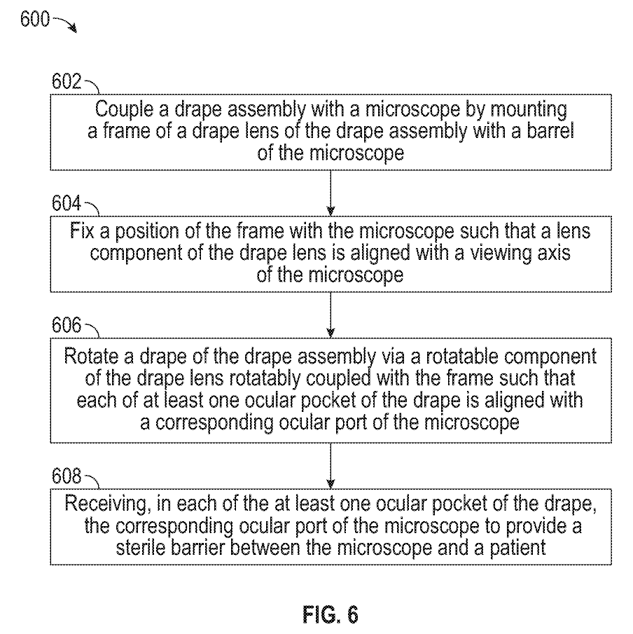

Referring to FIG. 6, a flowchart is presented in accordance with an example embodiment. The method 600 is provided by way of example, as there are a variety of ways to carry out the method. The method 600 described below can be carried out using the configurations illustrated in FIG. 1-5, for example, and various elements of these figures are referenced in explaining example method 1600. Each block shown in FIG. 6 represents one or more processes, methods or subroutines, carried out in the example method 600. Furthermore, the illustrated order of blocks is illustrative only and the order of the blocks can change according to the present disclosure. Additional blocks may be added or fewer blocks may be utilized, without departing from this disclosure. The example method 600 can begin at block 602.

At block 602, a drape assembly is coupled with a microscope by mounting a frame of a drape lens of the drape assembly with a barrel of the microscope.

At block 604, a position of the frame with the microscope is fixed such that a lens component of the drape lens is aligned with a viewing axis of the microscope.

At block 606, a drape of the drape assembly is rotated via a rotatable component of the drape lens rotatably coupled with the frame such that each of at least one ocular pocket of the drape is aligned with a corresponding ocular port of the microscope.

In at least one example, the drape is rotated about the drape lens so that each of the at least one ocular pocket is aligned with the corresponding ocular port of the microscope while the lens component remains stationary. In at least one example, the rotatable component includes a rotatable washer. In some examples, the rotatable washer can be coupled with the frame via at least one tab.

At block 608, each of the at least one ocular pocket of the drape receives the corresponding ocular port of the microscope to provide a sterile barrier between the microscope and a patient.

The disclosures shown and described above are only examples. Even though numerous properties and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

1. A drape lens for a drape assembly, the drape lens comprising:
a frame operable to be coupled with a microscope;
a lens component coupled with the frame; and
a rotatable component rotatably coupled with the frame, wherein the rotatable component is operable to be coupled with a drape.

2. The drape lens of claim 1, wherein the rotatable component includes a rotatable washer.

3. The drape lens of claim 2, wherein the rotatable washer is coupled with the frame via at least one tab.

4. The drape lens of claim 1, wherein the lens component is fixed in position when the frame is coupled with the microscope.

5. The drape lens of claim 1, wherein the frame includes at least one holding component extending therefrom, the at least one holding component operable to permit an operator to grasp onto the drape lens.

6. The drape lens of claim 1, wherein the lens component is operable to intersect at least one viewing axis of the microscope when the frame is coupled with the microscope.

7. A drape assembly comprising:
a drape including at least one ocular pocket, wherein each of the at least one ocular pocket is operable to correspond with a corresponding ocular port of a microscope;
a drape lens including:
a frame operable to be coupled with the microscope;
a lens component coupled with the frame; and
a rotatable component rotatably coupled with the frame,
wherein the rotatable component is operable to be coupled with the drape such that the drape is rotatable about the drape lens.

8. The drape assembly of claim 7, wherein the rotatable component includes a rotatable washer.

9. The drape assembly of claim 8, wherein the rotatable washer is coupled with the frame via at least one tab.

10. The drape assembly of claim 7, wherein the lens component is fixed in position when the frame is coupled with the microscope.

11. The drape assembly of claim 7, wherein the frame includes at least one holding component extending therefrom, the at least one holding component operable to permit an operator to grasp onto the drape lens.

12. The drape assembly of claim 7, wherein the lens component is operable to intersect at least one viewing axis of the microscope when the frame is coupled with the microscope.

13. The drape assembly of claim 7, wherein the at least one ocular pocket is operable to receive the corresponding ocular port of the microscope.

14. The drape assembly of claim 7, wherein the at least one ocular pocket includes three ocular pockets.

15. The drape assembly of claim 7, wherein the drape is operable to be rotatable about the drape lens so that each of the at least one ocular pocket is aligned with the corresponding ocular port of the microscope while the lens component remains stationary.

16. The drape assembly of claim 7, wherein the drape is operable to provide a sterile barrier between the microscope and a patient.

17. A method comprising:
coupling a drape assembly with a microscope by mounting a frame of a drape lens of the drape assembly with a barrel of the microscope;
fixing a position of the frame with the microscope such that a lens component of the drape lens is aligned with a viewing axis of the microscope;
rotating a drape of the drape assembly via a rotatable component of the drape lens rotatably coupled with the frame such that each of at least one ocular pocket of the drape is aligned with a corresponding ocular port of the microscope; and
receiving, by each of the at least one ocular pocket of the drape, the corresponding ocular port of the microscope to provide a sterile barrier between the microscope and a patient.

18. The method of claim 17, wherein the drape is rotated about the drape lens so that each of the at least one ocular pocket is aligned with the corresponding ocular port of the microscope while the lens component remains stationary.

19. The method of claim 17, wherein the rotatable component includes a rotatable washer.

20. The method of claim 19, wherein the rotatable washer is coupled with the frame via at least one tab.
